(19) United States
(12) Patent Application Publication
(10) Pub. No.: US 2024/0000607 A1
(43) Pub. Date: Jan. 4, 2024

(54) Negative Poisson's Ratio Materials for Thermal and Radiation Therapy Belts

(71) Applicant: Joon Bu Park, Las Vegas, NV (US)

(72) Inventor: Joon Bu Park, Las Vegas, NV (US)

(21) Appl. No.: 17/856,440

(22) Filed: Jul. 1, 2022

(51) Int. Cl. A61F 7/12; A61N 5/10

(52) U.S. Cl. CPC A61F 7/12; A61N 5/1001; A61F 2007/0001

(57) ABSTRACT

An implantable medical device includes a belt including an annular base extending around a circumference of the belt, in which the annular base defines an interior space of the belt that is sized to receive an internal organ of a patient. The annular base includes a biocompatible negative Poisson's ratio (NPR) foam material having a Poisson's ratio of between 0 and −1. In some cases, the annular base is configured to be inductively heated to thereby deliver thermal therapy to the organ. In some cases, the belt is a belt for brachytherapy that includes a radioactive material.

### BACKGROUND

The present disclosure relates generally to materials for belts for thermal and radiation therapy.

### SUMMARY

Thermal therapy and radiation therapy (e.g., brachytherapy) are non-invasive procedures to treat abnormal growths or tumors in the body (e.g., Benign Prostatic Hyperplasia (BPH), Adenocarcinoma of the prostate (CaP)). BPH is common in men and is classified by abnormal growth in the transition zone and periurethral tissue surrounding the urethra. As the adenomatous tissue within the transition zone expands, it can compress or block the urethra, causing irritation and obstruction. CaP, which is another prostate growth, is a commonly diagnosed cancer in the U.S. male population. Treatments for BPH and CaP can include surgery, medications, or thermal or radiation therapy. Thermal and radiation therapy can also be used to treat other types of abnormal growths or tumors.

Thermal therapy at moderate temperatures (e.g., about 41-45° C.) promotes changes in cellular dynamics. Immediate effects of thermal exposure in this temperature range include acceleration of metabolism, thermal inactivation of enzymes, and rupture of cell membranes. Other effects include intracellular and tissue edema as well as an increase in blood vessel permeability and dilatation. For low temperatures and shorter exposure times, the damage due to thermal effects alone can be reversible. For longer times or higher temperatures, cellular repair mechanisms can no longer keep up, or can lose function due to thermal damage of key enzymes, such that cell death and tissue necrosis can occur, e.g., within 3-5 days.

The localization of high-temperature hyperthermia at temperatures greater than about 45-50° C. can be used to selectively destroy or permanently alter tissue regions. In the high-temperature range, thermal coagulation and necrosis can occur in tissues exposed to temperatures greater than 50-55° C. for a duration of about 1-2 minutes or shorter times for even higher temperatures. Thermal exposure to these high temperatures can cause cellular and tissue structural proteins to undergo irreversible denaturation and conformational changes. These thermal effects can be lethal and immediate, producing thermally coagulated tissue. On the extreme end, temperatures close to or greater than 100° C. can cause ablation of tissue.

We describe here implants used to deliver thermal therapy or radiation therapy that include materials having a negative Poisson's ratio (“NPR materials”). NPR materials are lightweight and porous, and are capable of wrapping around and becoming embedded securely in the surrounding tissue due to their porosity and high surface area. Implants, such as thermal belts or radioactive belts for brachytherapy, can be formed of NPR materials alone or in conjunction with materials having a positive Poisson's ratio (“PPR materials”). In some examples, some portions of the belts are formed of NPR materials and other portions are formed of PPR materials. In some examples, composite materials that include both NPR materials and PPR materials are used for the belts.

In an aspect, an implantable medical device includes a belt including an annular base extending around a circumference of the belt, in which the annular base defines an interior space of the belt that is sized to receive an internal organ of a patient. The annular base includes a biocompatible negative Poisson's ratio (NPR) foam material having a Poisson's ratio of between 0 and −1.

Embodiments can include one or any combination of two or more of the following features.

The annular base is configured to be inductively heated to thereby deliver thermal therapy to the organ.

The belt includes a protrusion attached to or integral with the annular base, in which the protrusion extends outwardly from the circumference of the belt. In some cases, the implantable medical device includes multiple protrusions arranged at regular intervals around the circumference of the belt. In some cases, the protrusion includes a biocompatible NPR metal foam material.

The NPR material includes an NPR metal foam material. In some cases, the NPR metal foam material includes one or more of nickel, copper, palladium, or cobalt.

The NPR material is composed of a cellular structure having a characteristic dimension of between 0.01 μm and 3 mm.

The annular base includes a composite material including the NPR material and a positive Poisson's ratio (PPR) material. In some cases, the composite material includes alternating layers of the NPR material and the PPR material. In some cases, the alternating layers are oriented parallel to the circumference of the belt.

The annular base includes an inner layer and an outer layer covering the inner layer, and in which the outer layer includes the NPR material. In some cases, the inner layer includes a PPR material. In some cases, the interior layer includes a metal. In some cases, the belt includes a belt for brachytherapy, and in which the inner layer includes a radioactive material.

In an aspect, a method of providing a thermal treatment to an organ of a patient includes positioning a belt to surround the organ, the belt including: an annular base extending around a circumference of the belt, in which the annular base defines an interior space of the belt that is sized to receive an internal organ of a patient, and in which the annular base includes a biocompatible negative Poisson's ratio (NPR) foam material having a Poisson's ratio of between 0 and −1; and applying a magnetic field to the belt, in which application of the magnetic field induces an electrical current in each belt, the electrical current generating heat for the thermal treatment of the organ.

In an aspect, a method of treating a tumor with a brachytherapy treatment includes positioning a belt to surround a tumor, the belt including an annular base extending around a circumference of the belt, in which the annular base defines an interior space of the belt that is sized to receive an internal organ of a patient, in which the belt includes: an inner layer including a radioactive material, and an outer layer covering the inner layer, the outer layer including biocompatible negative Poisson's ratio (NPR) foam material having a Poisson's ratio of between 0 and −1; and allowing the implantable belts to remain in position surrounding the tumor for a predefined amount of time to deliver radiation to the tumor.

Other implementations are within the scope of the claims.

### DESCRIPTION OF DRAWINGS

FIGS. 1A and 1B are illustrations of a thermal belt.

FIG. 2 is an illustration of materials with negative and positive Poisson's ratios.

FIG. 3 is an illustration of composite materials.

FIG. 4 is an illustration of a material with a positive Poisson's ratio and a composite material.

FIGS. 5A and 5B are illustrations of thermal belts.

FIGS. 6A and 6B are illustrations of thermal belts.

FIG. 7 is an illustration of a radioactive belt.

FIG. 8 is an illustration of thermal seeds.

FIG. 9 is an illustration of belts implanted in a patient.

FIG. 10 is a diagram of a method of making an NPR material.

### DETAILED DESCRIPTION

We describe here implants used in thermal therapy (e.g., thermal belts) or radiation therapy (e.g., brachytherapy belts) that include materials having a negative Poisson's ratio (“NPR materials”). NPR materials are lightweight, porous, and capable of being embedded in the surrounding tissue more securely than conventional implants due to their porosity and greater surface area in contact with tissue. Implants can be formed of NPR materials alone or in conjunction with materials having a positive Poisson's ratio (“PPR materials”). In some examples, some portions of the belts are formed of NPR materials and other portions are formed of PPR materials. In some examples, composite materials that include both NPR materials and PPR materials are used for the belts.

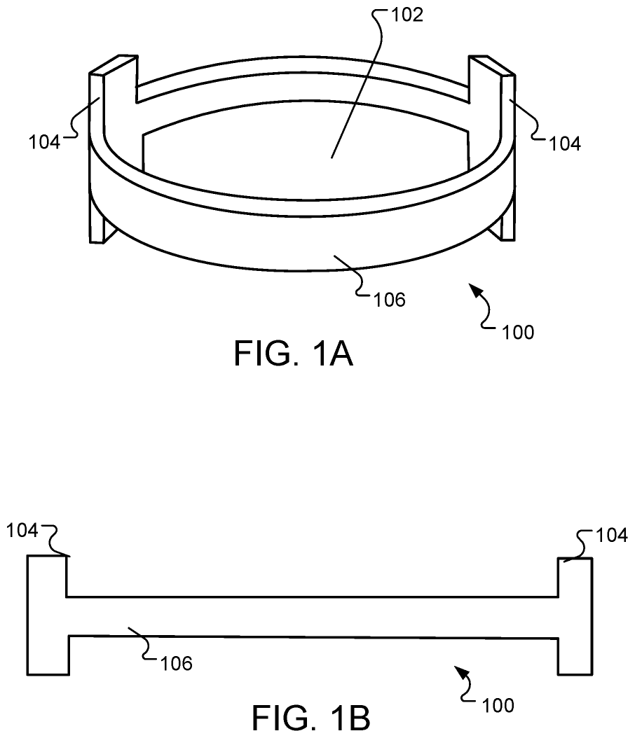

Referring to FIGS. 1A and 1B, an NPR thermal belt 100 is illustrated. The thermal belt 100 is rounded and has an opening 102, e.g., a generally circular or oval opening, that extends through the center of the NPR thermal belt 100. The NPR thermal belt 100 is placed around an organ (e.g., a prostate) of a patient such that the organ is disposed in the opening 102 and a base 106 of the thermal belt 100 circumferentially encircles the organ. The base 106 is an annular body, e.g., a circular, elliptical, or otherwise rounded body, that defines the circumference of the NPR thermal belt 100 and surrounds the opening 102. As discussed further below, the NPR thermal belt 100 is heated by induction heating, e.g., by application of a magnetic field to the NPR thermal belt. The induction-generated heat therapeutically heats the organ (e.g., prostate), e.g., for treatment of abnormal growths such as benign prostatic hyperplasia.

The NPR thermal belt 100 includes protrusions 104 which extend outwardly from the base 106 of the belt 100. In the illustrated example, the protrusions 104 extend in a direction perpendicular to the circumference of the belt 100; in some examples, the protrusions 104 extend outwardly from the base 106 at an angle that is less than perpendicular to the circumference. The protrusions 104 can be integral with the base 106 (e.g., formed of the same material and without a seam joining the protrusions with the base), or can be attached to the base (e.g., formed of the same or different material and joined to the base at a seam, weld, or other type of joint). In the illustrated example, the NPR thermal belt 100 includes two protrusions 104 disposed at diametrically opposed positions around the circumference of the belt 100. The protrusions 104 create additional surface area for contact with the organ (e.g., prostate) of the patient and help to stabilize the thermal belt on the organ. The protrusions can also be positioned to target delivery of thermal therapy to specific positions on the organ, e.g., by contacting the protrusions to target regions of the organ. In some implementations, the NPR thermal belt 100 can include more or fewer protrusions (e.g., 4, 3, 1, or 0 protrusions) than the two protrusions in the illustration. Although the protrusions 104 are illustrated as being evenly distributed around the belt 100 (e.g., at regular intervals around the circumference of the belt), in some implementations the protrusions 104 are distributed unevenly. The NPR thermal belt 100 is sized and shaped to be wrapped around an organ of a patient (e.g., a prostate of a patient). In some examples, the size of the NPR thermal belt 100 is adjustable.

In some examples, NPR thermal belts are formed of a biocompatible metal or metal alloy, such as nickel (Ni), copper (Cu), palladium (Pd), cobalt (Co), chromium (Cr), titanium (Ti), alloys of these or other metals (e.g., stainless steel, nitinol, NiCu, PdCo, CoCr), or other suitable metals or metal alloys. To use such thermal belts in, e.g., treatment of BPH, one or more thermal belts can be wrapped around the prostate of the patient. Once around the prostate, the thermal belts can be heated through, e.g., magnetic induction. Magnetic induction is the creation of electromagnetic forces (e.g., inducing an electrical current) using an electrical conductor (e.g., the NPR thermal belts 100) and changing magnetic fields. The induced electrical current in the thermal belts generates heat for application of thermal therapy to the tissue (e.g., prostate tissue) which contacts or is in the vicinity of the thermal belts.

The NPR thermal belts 100 described here are at least partially formed of a biocompatible NPR material (also referred to as an auxetic material) that is capable of inductive heating, e.g., an NPR metal. In some examples, the thermal belts 100 are formed completely of a biocompatible NPR material, such as a porous NPR material, e.g., a porous NPR metal material, meaning that the entirety of the thermal belt is formed of a biocompatible NPR material. In some examples, the porous material is an NPR foam material, e.g., an NPR metal foam, such as an NPR foam of a biocompatible metal or metal alloy, e.g., nickel, copper, palladium, cobalt, chromium, titanium, stainless steel, nitinol, or other suitable metals or metal alloys. In some examples, the thermal belts 100 are formed of a composite material that include both biocompatible NPR materials and biocompatible PPR materials, referred to as an NPR-PPR composite material. Forming the thermal belts 100 from an NPR material can have advantages. For instance, NPR materials, such as porous NPR materials, e.g., NPR foam materials, have a low density, and belts formed from NPR materials can be less obtrusive to a patient than comparable belts formed from only PPR materials. In addition, porous NPR materials, such as NPR foam materials, present a large surface area to the tissue contacted by the NPR belt, facilitating good contact between the belt and the tissue. This good contact can help with positional stability of the belt. The large area of contact between the foam morphology of the belt and the tissue also facilitates efficient heat transfer from the belt to the tissue, contributing to therapeutic effectiveness. In some embodiments, thermal belts can include or be formed from a mesh material to increase contact with the tissue.

An NPR material is a material that has a Poisson's ratio that is less than zero, such that when the material experiences a positive strain along one axis (e.g., when the material is stretched), the strain in the material along the two perpendicular axes is also positive (e.g., the material expands in cross-section). Conversely, when the material experiences a negative strain along one axis (e.g., when the material is compressed), the strain in the material along a perpendicular axis is also negative (e.g., the material compresses along the perpendicular axis). By contrast, a PPR material has a Poisson's ratio that is greater than zero. When a PPR material experiences a positive strain along one axis (e.g., when the material is stretched), the strain in the material along the two perpendicular axes is negative (e.g., the material compresses in cross-section), and vice versa.

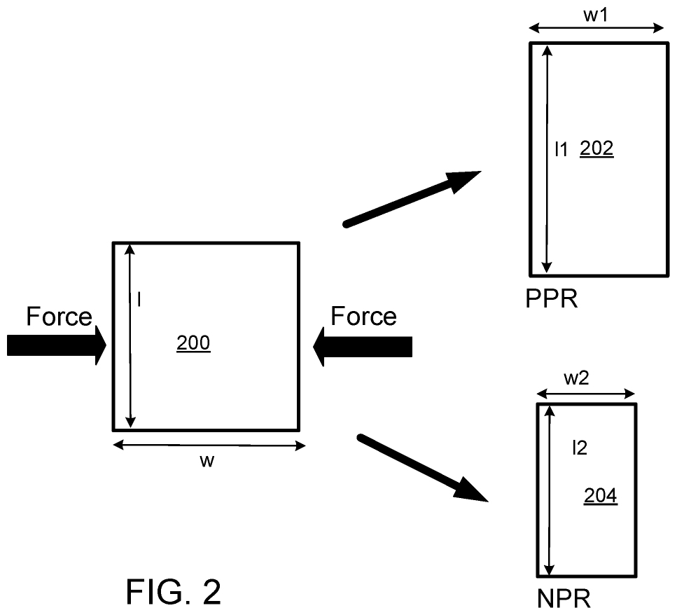

Materials with negative and positive Poisson's ratios are illustrated in FIG. 2, which depicts a hypothetical two-dimensional block of material 200 with length 1 and width w.

If the hypothetical block of material 200 is a PPR material, when the block of material 200 is compressed along its width w, the material deforms into the shape shown as block 202. The width w1 of block 202 is less than the width w of block 200, and the length 11 of block 202 is greater than the length 1 of block 200: the material compresses along its width and expands along its length.

By contrast, if the hypothetical block of material 200 is an NPR material, when the block of material 200 is compressed along its width w, the material deforms into the shape shown as block 204. Both the width w2 and the length 12 of block 204 are less than the width w and length 1, respectively, of block 200: the material compresses along both its width and its length.

NPR materials for thermal belts can be foams, such as polymeric foams, ceramic foams, metallic foams, or combinations thereof. A foam is a multi-phase composite material in which one phase is gaseous and the one or more other phases are solid (e.g., polymeric, ceramic, or metallic). Foams can be closed-cell foams, in which each gaseous cell is sealed by solid material; open-cell foams, in which the each cell communicates with the outside atmosphere; or mixed, in which some cells are closed and some cells are open.

An example of an NPR foam structure is a re-entrant structure, which is a foam in which the walls of the cells are concave, e.g., protruding inwards toward the interior of the cells. In a re-entrant foam, compression applied to opposing walls of a cell will cause the four other, inwardly directed walls of the cell to buckle inward further, causing the material in cross-section to compress, such that a compression occurs in all directions. Similarly, tension applied to opposing walls of a cell will cause the four other, inwardly directed walls of the cell to unfold, causing the material in cross-section to expand, such that expansion occurs in all directions. NPR foams can have a Poisson's ratio of between −1 and 0, e.g., between −0.8 and 0, e.g., −0.8, −0.7, −0.6, −0.5, −0.4, −0.3, −0.2, or −0.1. NPR foams can have an isotropic Poisson's ratio (e.g., Poisson's ratio is the same in all directions) or an anisotropic Poisson's ratio (e.g., Poisson's ratio when the foam is strained in one direction differs from Poisson's ratio when the foam is strained in a different direction).

An NPR foam can be polydisperse (e.g., the cells of the foam are not all of the same size) and disordered (e.g., the cells of the foam are randomly arranged, as opposed to being arranged in a regular lattice). An NPR foam can have a characteristic dimension (e.g., the size of a representative cell, such as the width of the cell from one wall to the opposing wall) ranging from 0.01 μm to about 3 mm, e.g., about 0.01 μm, about 0.05 μm, about 0.1 μm, about 0.5 μm, about 1 μm, about 10 μm, about 50 μm, about 100 μm, about 900 μm, about 1 mm, about 2 mm, or about 3 mm.

Examples of polymeric foams for thermal belts include thermoplastic polymer foams (e.g., polyester polyurethane or polyether polyurethane); viscoelastic elastomer foams; or thermosetting polymer foams such as silicone rubber. Examples of metallic foams include metallic foams based on Nickel (Ni), Copper (Cu), Palladium (Pd), Cobalt (Co), NiCu, PdCo, or other metals or alloys.

NPR-PPR composite materials are composites that include both regions of NPR material and regions of PPR material. NPR-PPR composite materials can be laminar composites, matrix composites (e.g., metal matrix composites, polymer matrix composites, or ceramic matrix composites), particulate reinforced composites, fiber reinforced composites, or other types of composite materials. In some examples, the NPR material is the matrix phase of the composite and the PPR material is the reinforcement phase, e.g., the particulate phase or fiber phase. In some examples, the PPR material is the matrix phase of the composite and the NPR material is the reinforcement phase.

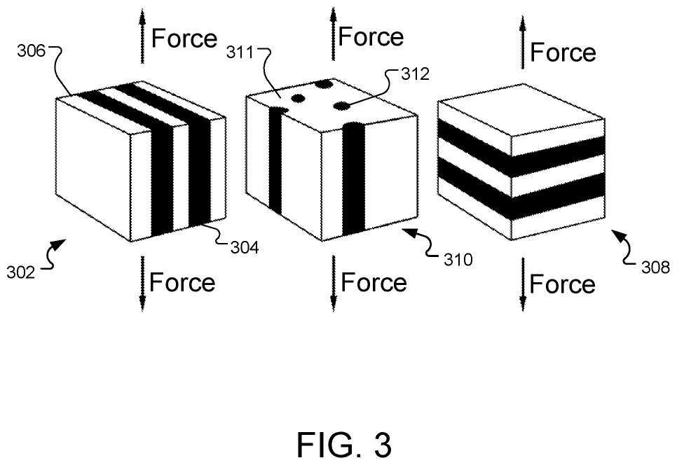

FIG. 3 illustrates examples of NPR-PPR composite materials. An NPR-PPR composite material 302 is a laminar composite including alternating layers 304 of NPR material and layers 305 of PPR material. The layers 304, 306 are arranged in parallel to a force to be exerted on the composite material 302. Although the layers 304, 306 are shown as having equal width, in some examples, a laminar composite can have layers of different widths.

An NPR-PPR composite material 308 is a laminar composite including alternating layers of NPR material and PPR material, with the layers arranged perpendicular to a force to be exerted on the material 308. In some examples, the layers of a laminar composite are arranged at an angle to the expected force that is neither perpendicular nor parallel.

An NPR-PPR composite material 312 is a matrix composite including a matrix phase 311 of NPR material with a reinforcement phase 312 of PPR material. In the material 312, the reinforcement phase 312 includes fibers of the PPR material; in some examples, the reinforcement phase 312 can include particles or other configuration. In some examples, NPR-PPR composite materials can have a matrix phase of a PPR material with a reinforcement phase of an NPR material.

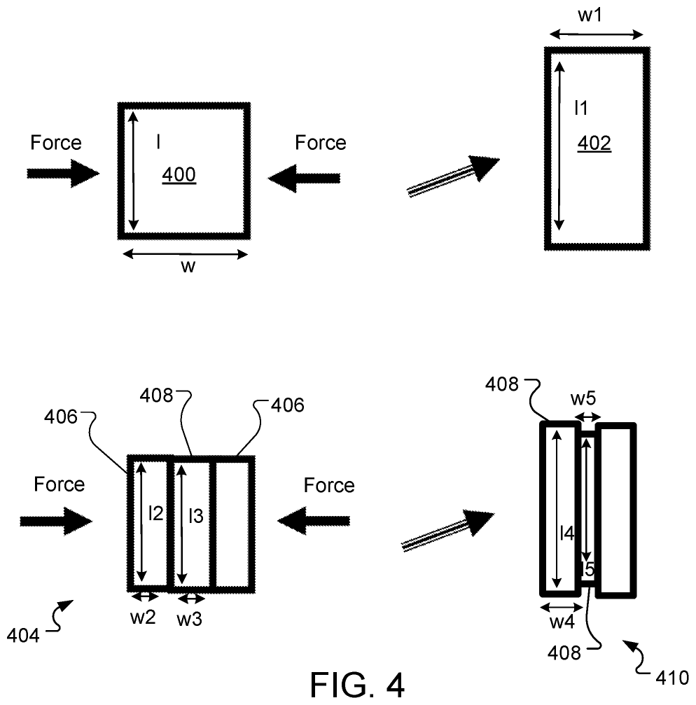

FIG. 4 illustrates the mechanical behavior of PPR and NPR/PPR composite materials. A hypothetical block 400 of PPR material, when compressed along its width w, deforms into a shape 402. The width w1 of the compressed block 402 is less than the width w of the uncompressed block 400, and the length 11 of the compressed block 402 is greater than the length 1 of the uncompressed block: the material compresses along the axis to which the compressive force is applied and expands along a perpendicular axis.

A block 404 of NPR/PPR composite material includes a region 408 of NPR material sandwiched between two regions 406 of PPR material. When the block 404 of composite material is compressed along its width, the material deforms into a shape 410. The PPR regions 406 compress along the axis of compression and expand along a perpendicular axis, e.g., as described above for the block 400 of PPR material, such that, e.g., the width w2 of a region 406 of uncompressed PPR material compresses to a smaller width w4 and the length 12 of the region 406 expands to a greater length 14. In contrast, the NPR region 408 compresses along both the axis of compression and along the perpendicular axis, such that, e.g., both the width w3 and length 13 of the uncompressed NPR region 408 are greater than the width w5 and length 15 of the compressed NPR region 408.

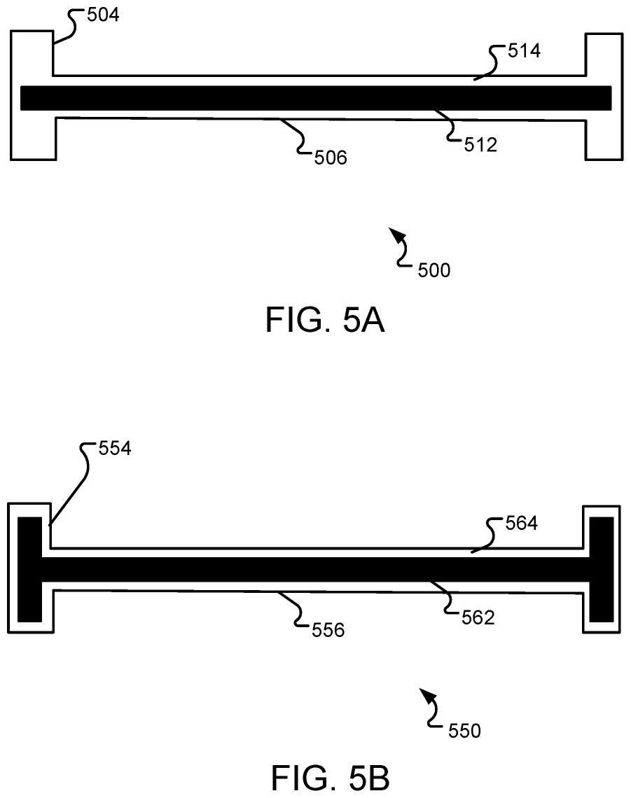

In some examples, NPR thermal belts can be multilayer structures. With reference to FIG. 5A, a base 506 of an NPR thermal belt 500 has an inner layer 512 and an outer layer 514. The outer layer 514 fully surrounds the inner layer 512 such that there is no exposure of the inner layer 512 to the external environment of the thermal belt 500, e.g., no contact between the inner layer 512 and the tissue, when the thermal belt 500 is implanted in a patient.

The inner layer 512 and the outer layer 514 are formed of different materials. The different composition of the inner layer 512 and outer layer 514 can allow for desired performance characteristics to be achieved. For instance, the inner layer 512 can be formed of a material with a high thermal capacity or thermal conductivity to facilitate effective thermal therapy, and the outer layer 514 can be formed of a biocompatible NPR material to provide a porous exterior that gives a large surface area in contact with the surrounding tissue. In some examples, the outer layer 514 is a non-porous material that acts as a barrier layer to prevent contact between the inner layer 512 and the surrounding tissue. In some examples, the outer layer 514 makes the thermal belt comfortable for the patient, e.g., the outer layer is formed of a low friction material (e.g., the material of the outer layer has a lower coefficient of friction than the material of the inner layer with respect to the patient's prostate), a softer material that conforms better to a patient's prostate than the material of the inner layer, or a moldable material. In an example, the outer layer 514 is formed of an NPR material, such as a porous NPR material, e.g., an NPR polymer foam or an NPR metal foam, or an NPR-PPR composite material; and the inner layer 512 is formed of a PPR material, such as a metal or metal alloy; or vice versa. A multi-layer NPR thermal belt having an outer layer composed of an NPR material and an inner layer composed of a material with a high thermal capacity can be advantageous because the belt can become embedded in the surrounding tissue, e.g., prostate, more securely due to the porosity and increased surface area in contact with the tissue provided by the NPR material, while still enabling efficient thermal treatment due to the high thermal capacity of the inner layer.

Referring to FIG. 5B, an NPR thermal belt 550 includes an inner layer 562 and an outer layer 564 that extend around a base 556 and along protrusions 554 of the thermal belt 550. The composition of the inner and outer layers 562, 564 can be as described for the thermal belt 500 of FIG. 5A.

In some examples, thermal belts are composite structures that have more than two layers (e.g., 3 layers, 4 layers, etc.). In implementations with more than two layers, each layer can be formed of a different material, or multiple layers can be formed of the same material. For instance, thermal belts can be constructed with alternating layers of NPR and PPR material, e.g., with an NPR material forming the outermost layer to provide a high surface area for contact with tissue.

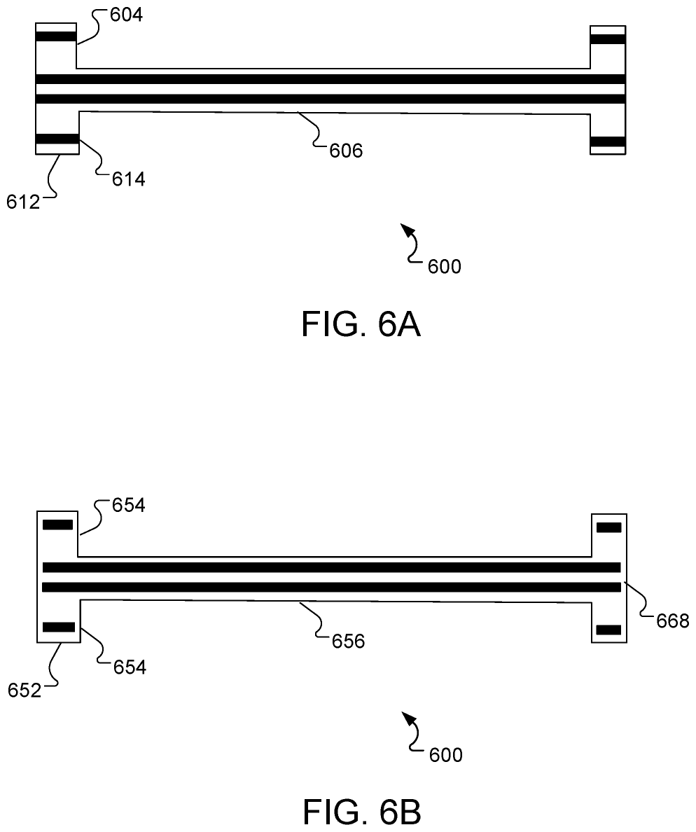

With reference to FIG. 6A, an NPR thermal belt 600 has NPR layers 612 and PPR layers 614 that extend circumferentially around a base 606 of the thermal belt 600 and along protrusions 604 of the thermal belt 600. In the illustrated example, the thermal belt 600 has five NPR layers 612 and four PPR layers 614, however in other embodiments thermal belts can have more or fewer layers. Both the NPR layers 612 and the PPR layers 614 extend to edges of the thermal belt 600 such that both NPR and PPR materials are exposed to an external environment, e.g., such that, when the belt is implanted in a patient, both NPR and PPR materials contact tissue of the patient. The PPR layers 614 have uniform thickness and the NPR layers 612 have different thicknesses, although in other embodiments the thicknesses of both the NPR layers 614 and the PPR layers 612 can have non-uniform thicknesses, and in some embodiments the thicknesses of all of the NPR layers 614 and the PPR layers 612 can be uniform.

Referring to FIG. 6B, an NPR thermal belt 650 includes NPR layers 662 and PPR layers 664, e.g., arranged as described for the thermal belt 600 of FIG. 6A. However, in the thermal belt 650 of FIG. 6B, the NPR and PPR layers 662, 664 do not extend all the way to the edges of the thermal belt 650. Rather, the thermal belt 650 also includes an outer region 668, e.g., of NPR material or PPR material, along the entire length of a base 656 of the belt 650 and along protrusions 654 of the belt. With the presence of the outer region 668, there is no exposure of the NPR and PPR layers 662, 664 to the external environment of the thermal belt 650, e.g., no contact between the layers 662, 664 and the tissue when the thermal belt 650 is implanted in a patient.

In some embodiments, the outer region 668 is the same material as the NPR layers 662. In some examples, the outer region 668 is the same material as the PPR layers 664. When the outer region 668 is an NPR material, the belt can become embedded in the surrounding tissue, e.g., prostate, more securely due to the porosity and increased surface area in contact with the tissue provided by the NPR material, while still enabling efficient thermal treatment due to the high thermal capacity of the composite NPR-PPR layers in the interior of the belt.

In some embodiments, the multiple layers of PPR and NPR materials can be arranged laterally rather than circumferentially, e.g., perpendicular to the circumference of the thermal belts. In some embodiments, the multiple layers of PPR and NPR materials can be arranged obliquely to the thermal belts, e.g., neither parallel to nor perpendicular to the circumference of the thermal belts.

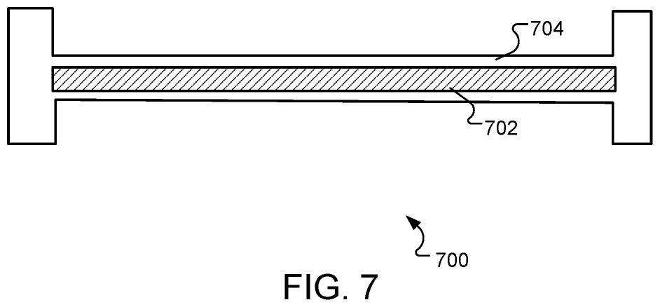

Radiation therapy (e.g., brachytherapy) can also utilize belts that include an NPR material. With reference to FIG. 7, an NPR radiotherapy belt 700 includes an outer layer 704 formed of an NPR material or an NPR-PPR composite material and an inner layer 702 formed of a radioactive material (e.g., radium, cesium, iridium, iodine, phosphorus, palladium). The inner layer 702 is partially or completely encapsulated within the outer layer 702. The outer layer 704 can be an NPR foam material, such as a porous NPR material, e.g., an NPR polymer foam, an NPR ceramic foam, or an NPR metal foam; or an NPR-PPR composite including multiple layers of NPR and PPR materials. An outer layer composed of an NPR material, such as a porous NPR material, e.g., an NPR foam material, can be advantageous because the belts can become embedded in the surrounding tissue, e.g., prostate, more securely due to the porosity and increased surface area in contact with the tissue. Some porous NPR materials, e.g., NPR foam materials, are relatively soft, moldable, or both, allowing the belts to conform well to the surrounding tissue, which helps ensure positional stability. In some examples, the porous NPR material, e.g., NPR foam material, of the outer layer 704 can be low friction material (e.g., the outer layer has a lower coefficient of friction than the inner layer with respect to the patient's prostate), which can help make the insertion of the belts 700 less irritating to the patient's tissue.

Using radioactive belts in a brachytherapy procedure can allow a higher dose of radiation to be delivered to a limited area than conventional, external beam radiation treatments. Moreover, the shape of the belt can help the belt stay in a targeted position on an organ. Delivery of brachytherapy treatments using radioactive, NPR belts can be more effective at destroying cancer cells than conventional radiation treatments while minimizing damage to surrounding normal tissue. The radiation emitted from the inner layer 702 can kill damaging tumors or cells (e.g., cancer cells). The outer layer 704 is formed of a material that does not interfere with (e.g., does not absorb) the radiation emitted from the inner layer 702.

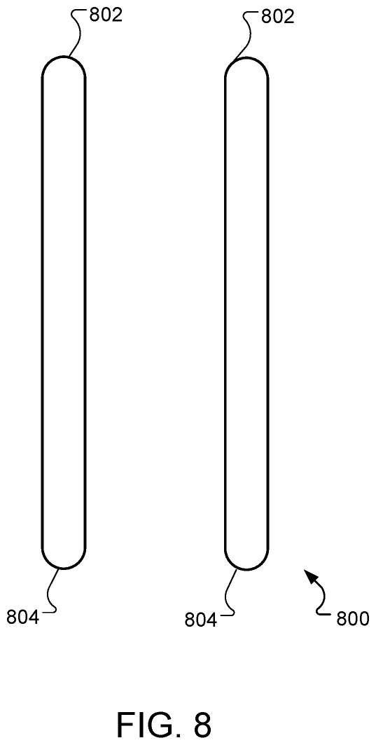

FIG. 8 illustrates thermal seeds 800. The thermal seeds 800 have an elongated body that is sized and shaped to be at least partially inserted into a patient (e.g., a prostate of a patient). For instance, the elongated body of the thermal seeds 800 can be generally cylindrical, e.g., with a diameter of about 1 millimeters (mm) and a length of about 10 mm. Although the thermal seeds are illustrated in a generally cylindrical shape, the thermal seeds can be a variety of shapes that fill the prostate (e.g., hexagonal, octagonal, elliptical, etc.). Additionally, the thermal seeds can have a variable diameter from a proximal end 802 to a distal end 804.

In some examples, thermal seeds are formed of a biocompatible metal or metal alloy, such as Nickel (Ni), Copper (Cu), Palladium (Pd), Cobalt (Co), NiCu, PdCo, or other suitable materials. To use such thermal seeds in, e.g., treatment of BPH, the thermal seeds are inserted into the prostate of the patient. Once in the prostate, the thermal seeds can be heated through, e.g., magnetic induction. Magnetic induction is the creation of electromagnetic forces (e.g., inducing an electrical current) using an electrical conductor (e.g., the thermal seeds 800) and changing magnetic fields. The induced electrical current in the thermal seeds can create heat for application of thermal therapy to the tissue in which the seeds are implanted, e.g., to the prostate. The thermal seeds 800 described here can be at least partially formed of a biocompatible NPR material (also referred to as an auxetic material). Such thermal seeds are described in U.S. application Ser. No. 17/345,486, filed Jun. 11, 2021, which is incorporated herein by reference in its entirety.

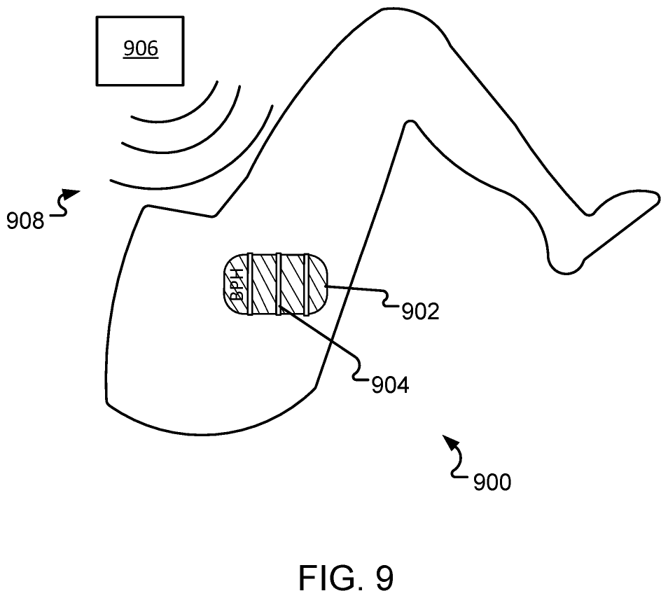

FIG. 9 illustrates thermal belts 904 wrapped around a prostate 902 of a patient 900. A magnetic field source 906 applies a magnetic field 908 to the belts, inducing electromagnetic forces in the belts 904 through magnetic induction. As described above, magnetic induction is the creation of electromagnetic forces (e.g., inducing an electrical current) using an electrical conductor (i.e., thermal belts) and changing magnetic fields. The induced electrical current in the belts generates heat for delivery of thermal therapy of the surrounding tissue, e.g., the prostate 902. In some implementations, thermal seeds (e.g., similar to thermal seeds 800) can be implanted in the prostate 902 concurrent with implantation of the bels 904 to provide additional thermal therapy.

In the case of brachytherapy, the implanted belts are radioactive and emit radiation, and the magnetic field source 906 is not used. The belts are implanted in the tissue, e.g., in a tumor, and allowed to remain in the tumor for a predetermined amount of time sufficient to deliver a clinically meaningful amount of radiation to the tumor. In some implementations, radioactive seeds can be implanted in the prostate 902 to provide additional brachytherapy.

In some examples, porous NPR materials, such as NPR foams, are produced by transformation of PPR foams to change the structure of the foam into a structure that exhibits a negative Poisson's ratio. In some examples, porous NPR materials, such as NPR foams, are produced by transformation of nanostructured or microstructured PPR materials, such as nanospheres, microspheres, nanotubes, microtubes, or other nano- or micro-structured materials, into a foam structure that exhibits a negative Poisson's ratio. The transformation of a PPR foam or a nanostructured or microstructured material into an NPR foam can involve thermal treatment (e.g., heating, cooling, or both), application of pressure, or a combination thereof. In some examples, PPR materials, such as PPR foams or nanostructured or microstructured PPR materials, are transformed into NPR materials by chemical processes, e.g., by using glue. In some examples, NPR materials are fabricated using micromachining or lithographic techniques, e.g., by laser micromachining or lithographic patterning of thin layers of material. In some examples, NPR materials are fabricated by additive manufacturing (e.g., three-dimensional (3D) printing) techniques, such as stereolithography, selective laser sintering, or other appropriate additive manufacturing technique.

In an example, a PPR thermoplastic foam, such as an elastomeric silicone film, can be transformed into an NPR foam by compressing the PPR foam, heating the compressed foam to a temperature above its softening point, and cooling the compressed foam. In an example, a PPR foam composed of a ductile metal can be transformed into an NPR foam by uniaxially compressing the PPR foam until the foam yields, followed by uniaxially compression in other directions.

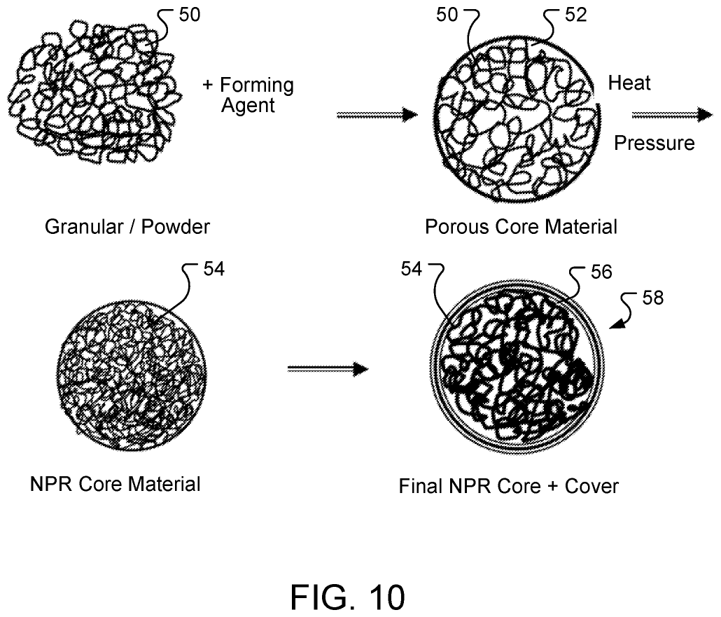

FIG. 10 illustrates an example method of making a multi-layer thermal belt in which an inner layer is formed of an NPR material. A granular or powdered material, such as a polymer material (e.g., a rubber) is mixed with a foaming agent to form a porous material 50. The porous material 50 is placed into a mold 52. Pressure is applied to compress the material 50 and the compressed material is heated to a temperature above its softening point. The material is then allowed to cool, resulting in a porous NPR material 54. The porous NPR material 54 is covered with an outer layer 56, such as a polymer layer, and heat and pressure can be applied again to cure the final material into a thermal belt 58.

In some examples, such as for brachytherapy belts, the outer layer of the belt is formed of an NPR material. A granular or powdered material, such as a polymer material (e.g., a rubber) is mixed with a foaming agent to form a porous material. The porous material is placed into a mold surrounding an inner layer (e.g., a radioactive layer). Pressure is applied to compress the material and the compressed material is heated to a temperature above its softening point. The material is then allowed to cool, resulting in a porous NPR outer layer.

Other methods can also be used to fabricate thermal belts or brachytherapy belts formed of an NPR material or an NPR-PPR composite material. For example, various additive manufacturing (e.g., 3D printing) techniques, such as stereolithography, selective laser sintering, or other appropriate additive manufacturing technique, can be implemented to fabricate an thermal belt formed of an NPR material or an NPR-PPR composite. In some examples, different components of the thermal belt are made by different techniques. For example, the inner layer may be 3D printed while the outer layer is not, or vice versa. Additive manufacturing techniques can enable seams to be eliminated.

Particular embodiments of the subject matter have been described. Other embodiments are within the scope of the following claims.

What is claimed is:

1. An implantable medical device comprising:
a belt comprising an annular base extending around a circumference of the belt, in which the annular base defines an interior space of the belt that is sized to receive an internal organ of a patient,
in which the annular base comprises a biocompatible negative Poisson's ratio (NPR) foam material having a Poisson's ratio of between 0 and −1.

2. The implantable medical device of claim 1, in which the annular base is configured to be inductively heated to thereby deliver thermal therapy to the organ.

3. The implantable medical device of claim 1, in which the belt comprises a protrusion attached to or integral with the annular base, in which the protrusion extends outwardly from the circumference of the belt.

4. The implantable medical device of claim 3, comprising multiple protrusions arranged at regular intervals around the circumference of the belt.

5. The implantable medical device of claim 3, in which the protrusion comprises a biocompatible NPR metal foam material.

6. The biocompatible belt of claim 1, in which the NPR material comprises an NPR metal foam material.

7. The implantable medical device of claim 6, in which the NPR metal foam material comprises one or more of nickel, copper, palladium, or cobalt.

8. The implantable medical device of claim 1, in which the NPR material is composed of a cellular structure having a characteristic dimension of between 0.01 μm and 3 mm.

9. The implantable medical device of claim 1, in which the annular base comprises a composite material comprising the NPR material and a positive Poisson's ratio (PPR) material.

10. The implantable medical device of claim 9, in which the composite material comprises alternating layers of the NPR material and the PPR material.

11. The implantable medical device of claim 10, in which the alternating layers are oriented parallel to the circumference of the belt.

12. The implantable medical device of claim 1, in which the annular base comprises an inner layer and an outer layer covering the inner layer, and in which the outer layer comprises the NPR material.

13. The implantable medical device of claim 12, in which the inner layer comprises a PPR material.

14. The implantable medical device of claim 12, in which the interior layer comprises a metal.

15. The implantable medical device of claim 12, in which the belt comprises a belt for brachytherapy, and in which the inner layer comprises a radioactive material.

16. A method of providing a thermal treatment to an organ of a patient, the method comprising:
positioning a belt to surround the organ, the belt comprising:
an annular base extending around a circumference of the belt, in which the annular base defines an interior space of the belt that is sized to receive an internal organ of a patient, and
in which the annular base comprises a biocompatible negative Poisson's ratio (NPR) foam material having a Poisson's ratio of between 0 and −1; and
applying a magnetic field to the belt, in which application of the magnetic field induces an electrical current in each belt, the electrical current generating heat for the thermal treatment of the organ.

17. A method of treating a tumor with a brachytherapy treatment, the method comprising:
positioning a belt to surround a tumor, the belt comprising an annular base extending around a circumference of the belt, in which the annular base defines an interior space of the belt that is sized to receive an internal organ of a patient, in which the belt comprises:
an inner layer comprising a radioactive material, and
an outer layer covering the inner layer, the outer layer comprising biocompatible negative Poisson's ratio (NPR) foam material having a Poisson's ratio of between 0 and −1; and
allowing the implantable belts to remain in position surrounding the tumor for a predefined amount of time to deliver radiation to the tumor.
